(19) United States
(12) Patent Application Publication
(10) Pub. No.: US 2010/0132097 A1
(43) Pub. Date: Jun. 3, 2010

(54) STRUCTURE FOR SAFETY HELMET

(76) Inventor: Tsan-Jee Chen, Taipei (TW)

Correspondence Address: PAI PATENT & TRADEMARK LAW FIRM, 1001 FOURTH AVENUE, SUITE 3200, SEATTLE, WA, 98154, US

(21) Appl. No.: 12/431,002

(22) Filed: Apr. 28, 2009

Related U.S. Application Data
(63) Continuation-in-part of application No. 12/327,810, filed on Dec. 3, 2008.

(51) Int. Cl. A42B 3/22; A61F 9/00

(52) U.S. Cl. 2/425; 2/12

(57) ABSTRACT

The safety helmet structure comprises a main body, an outer visor, an inner visor assembly including an inner visor and two moveable seats, a connective portion, a seating unit and a push seat. A connective part is disposed on both sides of the inner visor. The two moveable seats are connected with the connective parts through slots. A position-limiting slot is provided on the outer side of the moveable seat. A hole is provided on the rear part of the moveable seat. The moveable seat may be connected with the helmet through the hole. The moveable seat is connected with the connective portion through the connective hole. The inner visor may be retracted into the helmet or extended out by merely pushing the push seat.

This application is a continuation-in-part of U.S. patent application Ser. No. 12/327,810, filed on Dec. 3, 2008, now pending.

### BACKGROUND OF THE INVENTION

1. Field of the Invention

The invention generally relates to a structure used for safety helmets. More particularly, the invention relates to a safety helmet structure which provides a retractable inner visor that may be fitted onto a safety helmet to block the sunlight.

2. Description of the Prior Art

Most of the visors fitted on the safety helmets on the market are transparent to allow riders to see clearly. If the sunlight becomes too strong or shines directly into the eyes, many riders would wear sunglasses to weaken the sunlight; alternatively, some riders may replace the transparent visor with a light-reflective visor or a colored visor.

Though the sunlight is weakened in either way mentioned above, it would be difficult for a rider to take out his sunglasses from his bag when his motorcycle is in motion. Also, though a colored visor can effectively block the sunlight, such visor would make it difficult for him to see clearly during the nighttime and may even cause an accident.

From the above, we can see that the safety helmet of the prior art has many disadvantages and needs to be improved.

To eliminate the disadvantages of the safety helmet of the prior art, the inventor has put in a lot of effort in the subject and has successfully come up with the safety helmet structure of the present invention.

### SUMMARY OF THE INVENTION

The object of the present invention is to provide a retractable inner visor that may be fitted onto a helmet to block or weaken the sunlight.

The safety helmet structure of the present invention comprises a main body, an outer visor, an inner visor assembly, two moveable seats, a connective portion, a seating unit and a push seat. The two ends of the outer visor are connected with two first connective sections of the main body. A position-limiting protrusion and a second connective section are disposed on either side of the inside of the main body. The inner visor assembly may be connected to the main body through the holes. A connective part is disposed on both sides of the inner visor. A connective element is disposed on each connective part. The two moveable seats are connected with the connective parts of the inner visor through slots. A position-limiting slot is provided on the outer side of the moveable seat. Each position-limiting protrusion is held in the corresponding position-limiting slot. A connective hole is provided at a position near the position-limiting slot. Several indentations may be provided on the connective hole. A hole is provided on the rear part of the moveable seat. The moveable seat may be connected with the helmet through the hole. A shaft is disposed on the inner side of the connective part and at least one protrusion is disposed on the shaft of the connective portion. A connective hole is provided on the other side of the connective portion. The connective portion may be connected with the connective hole of the moveable seat through the shaft. The seat unit is fixedly held on the outer side of the helmet through the fastening protrusion. A protrusion is disposed on the central part of the inner side of the push seat and may engage with a slot of the seating unit. At least one engagement element is disposed at the front end of the protrusion and may fixedly hold the push seat in position. Also, the front part of the protrusion may be inserted into the connective hole of the connective portion, and a fastener can be used to affix it. Therefore, the connective portion may be connected with the protrusion of the push seat. Then, the assembled components are fitted to a main body. By pushing the push seat forward or backward, a user may extend or retract the inner visor through the inter-linking of the components.

These features and advantages of the present invention will be fully illustrated and explained in the following detailed description in view of the accompanying Drawings.

### BRIEF DESCRIPTION OF THE DRAWINGS

FIG. 1 is a side view of the safety helmet structure of the present invention.

FIG. 2 is an exploded perspective view showing the components of the safety helmet structure of the present invention.

FIG. 3 is a perspective view showing the components of the safety helmet structure of the present invention in an assembled condition.

FIGS. 4A and 4B are two side views illustrating how the safety helmet structure of the present invention is used after it is fitted to a main body.

### DETAILED DESCRIPTION OF THE PREFERRED EMBODIMENT

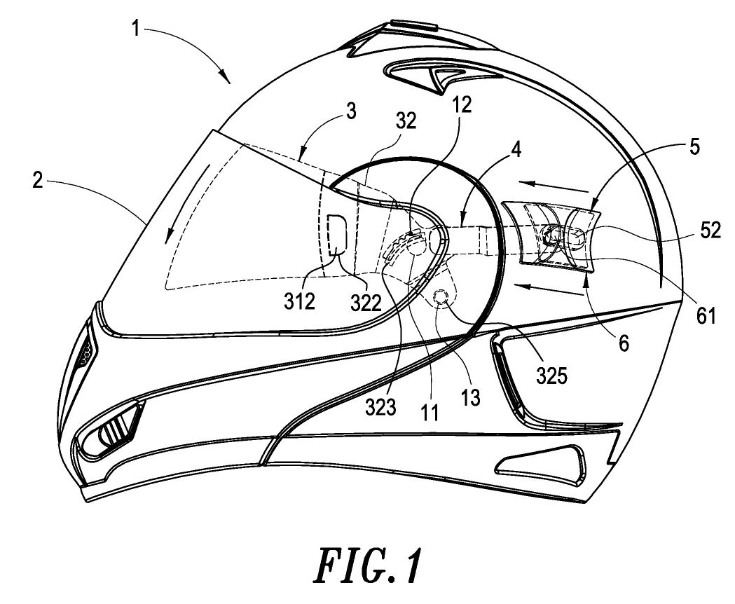

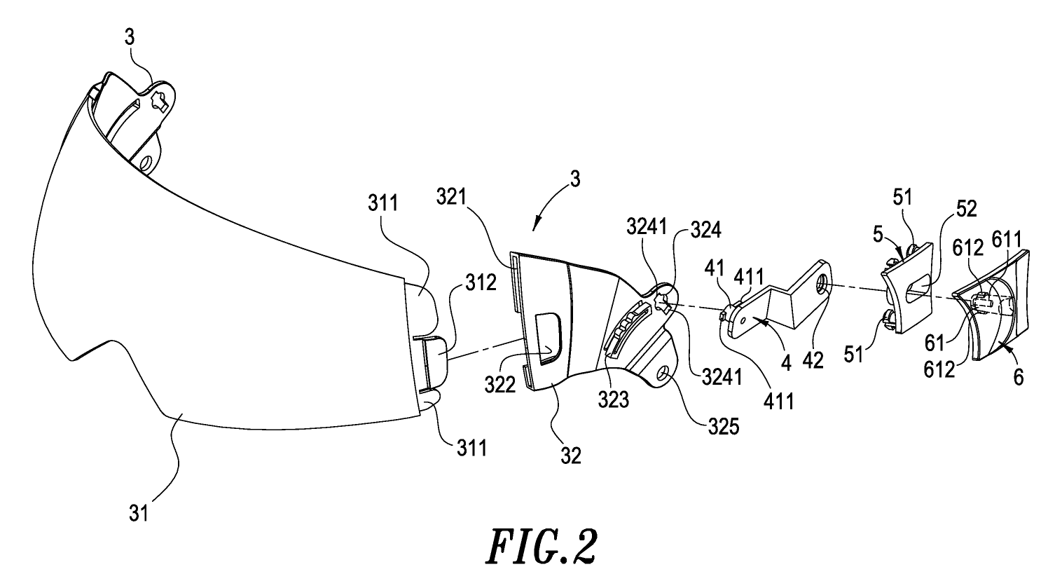

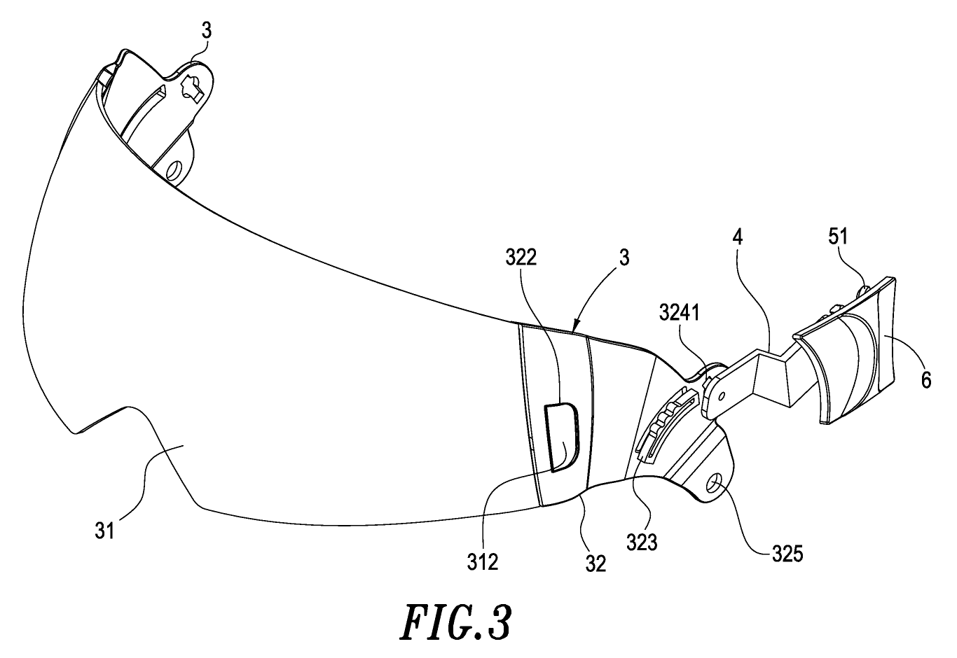

As illustrated in FIGS. 1 to 4B, the safety helmet structure of the present invention comprises a main body 1, an outer visor 2, an inner visor assembly 3, a connective portion 4, a seating unit 5 and a push seat 6.

A first connective section 11 is disposed on each of two sides of the outside of the main body 1. A position-limiting protrusion 12 and a second connective section 13 are disposed on each of two sides of the inside of the main body 1.

The two ends of the outer visor 2 are connected with the two first connective sections 11. The purpose of the outer visor 2 is to block the wind and airborne dust/particles.

The inner visor assembly 3 comprises an inner visor 31 and two moveable seats 32. A connective part 311 is disposed on each of two ends of the inner visor 31, and a connective element 312 is disposed on each connective part 311. A slot 321 is provided on the front end of each moveable seat 32 to receive the corresponding connective part 311 and a hole 322 is provided in each slot 321 to receive the connective element 312. A position-limiting slot 323 is disposed on the outer side of each moveable seat 32. The front side of the position-limiting slot 323 has a saw-toothed shape. A connective hole 324 is provided at a position near the position limiting slot 323, and at least an indentation 3241 is provided in the connective hole 324. A hole 325 is provided on the rear part of the moveable seat 32 so that the second connective section 13 on the inner side of the main body 1 may mate with the inner visor assembly 3 via the hole 325. In addition, the upward or downward movement of the outer visor 2 does not have any bearing on the inner visor assembly 3. The position-limiting protrusion 12 of the main body 1 may be held inside the position-limiting slot 323. The purpose of the inner visor 3 is to block or weaken the sunlight.

A shaft 41 is disposed on the inner side of the connective portion 4. At least one protrusion 411 is disposed on the shaft 41. The shaft 41 and the protrusion 411 may engage with the corresponding connective hole 324 and indentation 3241 of the corresponding moveable seat 32. Also, a connective hole 42 is provided on the other side of the connective portion 4.

At least one fastening protrusion 51 is disposed on the inner side of the seating unit 5. A slot 52 is provided on the central part of the seating unit 5. Each seating unit 5 is affixed to the outside of the main body 1 through the fastening protrusion 51.

A protrusion 61 is disposed on the central part of the inner side of the push seat 6. A threaded hole 611 is provided on the end of the protrusion 61. At least one engagement element 612 is disposed at the front end of the protrusion 61. The protrusion 61 may engage with the slot 52 of the seating unit 5, and the engagement element 612 may press against the slot 52; therefore, the push seat 6 may connect with the seating unit 5. Also, the front part of the protrusion 61 may be inserted into the connective hole 42, and a fastener (not shown) can be affixed to the threaded hole 611. Therefore, the connective portion 4 may be connected with the protrusion 61. The said fastener may be a screw.

In assembly, first, the aforementioned components are assembled; then, the assembled components are fitted to the main body of a safety helmet.

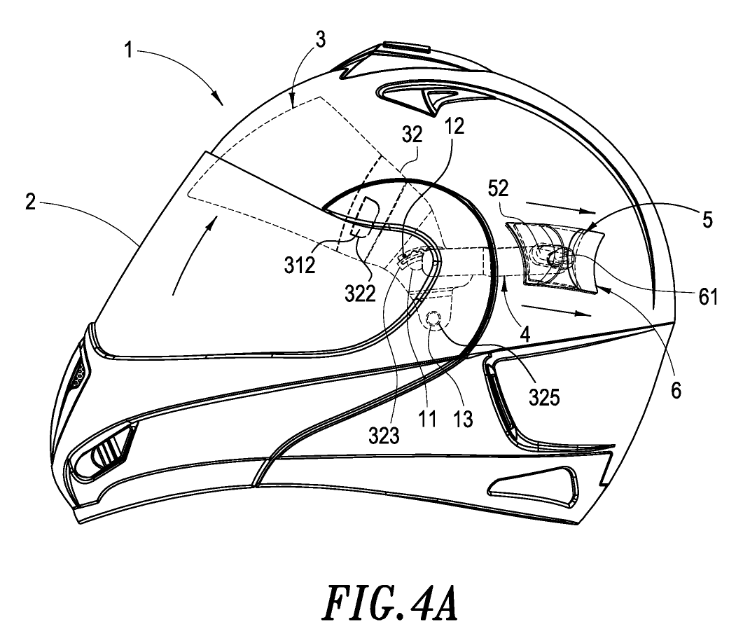

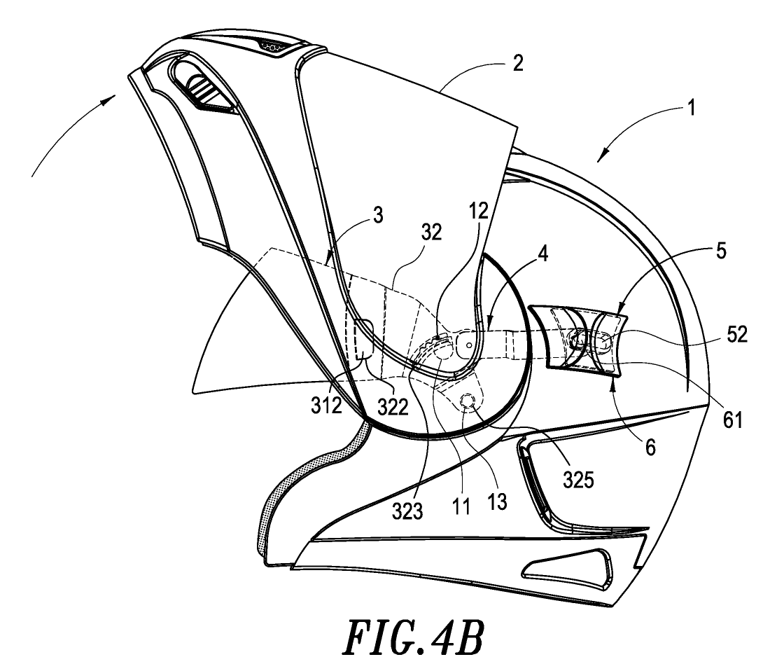

Now, please refer to FIGS. 4A and 4B, which show how the structure of the present invention is used. When the user wants to retract the inner visor assembly 3, he only has to push the push seat 6 backward. Because the protrusion 61 is linked with the connective portion 4, the connective portion 4 is moved backward. Furthermore, because one end of each moveable seat 32 is affixed to the inner surface of the main body 1, the inner visor assembly 3 is pushed upward by the connective portion 4, as illustrated in FIG. 4A. Now, the inner visor assembly 3 is retracted into the main body 1. When the user wants to extend the inner visor assembly 3, he only has to push the push seat 6 forward. Because the protrusion 61 is linked with the connective portion 4, the connective portion 4 is moved forward. Also, because one end of each moveable seat 32 is affixed to the inner surface of the main body 1, the inner visor assembly 3 is pushed downward by the connective portion 4, as illustrated in FIG. 4B. Now, the inner visor assembly 3 is extended and the sunlight may thus be blocked by the visor 1. In addition, the upward or downward movement of the outer visor 2 does not have any bearing on the inner visor assembly 3.

A position-limiting protrusion 12 is disposed on either side on the inside of the main body 1 and is held in the corresponding position-limiting slot 323. Because the front side of each position-limiting slot 323 has a saw-toothed shape, several positions may be selected.

In contrast to the helmet of the prior art, the helmet structure of the present invention has the following advantage that the retractable inner visor may be fitted onto a safety helmet and can be easily extended out to block the sunlight.

Many changes and modifications in the above described embodiment of the invention can, of course, be carried out without departing from the scope thereof. Accordingly, the invention is disclosed and should be limited only by the scope of the appended claims.

What is claimed is:

1. A structure for safety helmet, comprising:
a main body, having a first connective section disposed on either side of the outside of the main body and having a position-limiting protrusion and a second connective section disposed on either side of the inside of the main body;
an outer visor, the two ends of which being connected with the two first connective sections of the main body;
an inner visor assembly, comprising an inner visor and two moveable seats, a connective part being disposed on either end of the inner visor assembly, and a connective element being disposed on each connective part, a slot being provided on the front end of each moveable seat and a hole being provided in each slot to receive the corresponding connective part (the connective element being received by the corresponding hole), a position-limiting slot being disposed on the outer side of each moveable seat, the inner part of the position limiting slot having a saw-toothed shape, a connective hole being provided at a position lower to the position limiting slot, and at least an indentation being provided in the connective hole, a hole being provided on the rear part of the moveable seat, the second connective section and thus the inner visor assembly being able to mate with the hole, the upward or downward movement of the outer visor not having any bearing on the inner visor assembly, the position-limiting protrusion of the main body being able to be held inside the position-limiting slot, the purpose of the inner visor being to block or weaken the sunlight;
a connective portion, a shaft being disposed on the inner side of each connective portion, a connective hole being provided on the other side of the connective portion and the shaft engaging with the corresponding hole of the moveable seat;
a seating unit, at least one fastening protrusion being disposed on the inner side of each seating unit, a slot being provided on the central part of the seating unit and the seating unit being affixed to the helmet through the corresponding fastening protrusion; and
a push seat, a protrusion being disposed on the central part of the inner side of the push seat, a threaded hole being provided on the end of the protrusion, at least one engagement element being disposed at the front end of the protrusion, the protrusion engaging with the slot of the seating unit, the engagement element pressing against the slot, the push seat connecting with the corresponding seating unit, the front part of the protrusion being inserted into the connective hole, and a fastener being used to affix it with the threaded hole and thus the connective portion being connected with the protrusion,
characterized in that, in assembly, first, the aforementioned components being assembled and then the assembled components being fitted to a safety helmet so as to allow the upward and downward motions of the inner visor.

2. The safety helmet structure as in claim 1, wherein several indentations are provided on the connective hole of the moveable seat on the same side of the connective portion and several protrusions are provided on the shaft of the connective portion so that the connective hole and the indentations of the moveable seat may engage and fixedly connect with the shaft and the protrusions of the connective portion.

3. The safety helmet structure as in claim 1, wherein the fastener is a screw.
